(19) United States
(12) Patent Application Publication
(10) Pub. No.: US 2003/0232358 A1
(43) Pub. Date: Dec. 18, 2003

(54) Method of identifying genes controlling differentiation

(76) Inventor: James A. Thomson, Madison, WI (US)

Correspondence Address: Nicholas J. Seay, Quarles & Brady LLP, P O Box 2113, Madison, WI, 53701-2113, US

(21) Appl. No.: 10/389,120

(22) Filed: Mar. 14, 2003

Related U.S. Application Data
(60) Provisional application No. 60/365,359, filed on Mar. 15, 2002.

(51) Int. Cl. C12Q 1/68; G01N 33/53; G01N 33/567; C12N 5/08; C12N 15/867

(52) U.S. Cl. 435/6; 435/7.2; 435/455; 435/456; 435/366

(57) ABSTRACT

A method for identifying the genetic factors responsible for cell differentiation based on expression cloning is described. To determine what genetic factor leads to the differentiation of a cell from a beginning cell type to a target cell type, a cDNA library is obtained, packaged in an expression vector and transformed into cells of the beginning cell type. The expression vector also preferable includes a marker gene system under the control of a tissue specific promoter operable in the tissue type of the target cell type. The transformed cells are then cultured until cell differentiation begins, then the culture is screened or selected to identify the cells which undergo differentiation toward the target cell type. By examining the cDNA insert in the differentiated cells, the genetic factor responsible for the differentiation process can be identified.

### CROSS-REFERENCE TO RELATED APPLICATIONS

This application claims priority from U.S. provisional patent application S. No. 60/365,359 filed Mar. 15, 2002.

### STATEMENT REGARDING FEDERALLY SPONSORED RESEARCH OR DEVELOPMENT

Not applicable.

### BACKGROUND OF THE INVENTION

Modem cell biology includes a variety of techniques to manipulate various cells of living organisms in vitro. A particularly significant and intriguing category of cell culture are known as stem cells. Stem cells are undifferentiated, or only partially differentiated cells, that have the capability to differentiate into a number of progenitor cell types. The term stem cells can be used to refer to a cell type which is the progenitor of a category of a cell type in a larger organism, such as a hematopoietic stem cell, or can refer to a totally undifferentiated stem cell which, at least in theory, has the ability to differentiate into any of the tissues of the body of the whole organism. Stem cell cultures have been developed from a variety of tissues and in a number of different animals.

Recently it has become possible to generate, culture, and maintain cultures of primate embryonic stem cells, including human embryonic stem cells. For example, see U.S. Pat. Nos. 5,843,780 and 6,200,806 to Thomson. Primate embryonic stem cells are stem cell cultures, originally created from cells taken from embryos, that survive indefinitely in culture and are made up of cells which have the capability of differentiating into the major tissue types of a primate body. Primate embryonic stem cells can be maintained in an undifferentiated state in culture, or can be allowed to begin a differentiation process by which the cells become committed to one or another developmental cell lineage. Typically the differentiation of stem cells into different tissue types begins with the creation of embryoid bodies, which causes the stem cells in the embryoid body to begin to differentiate into different cell types in different portions of the embryoid body. In fact, maintaining human embryonic stem cells in an undifferentiated state requires careful attention to culture conditions since the cells will spontaneously begin uncontrolled differentiation if the culture conditions are incorrect.

One of the significant areas of research enabled by the development of stem cells is to begin to try to understand what genes or factors cause undifferentiated cells to begin to differentiate into committed cell lineages. It is theorized that for most initial differentiation of stem cells that a single genetic factor turns on, or off, causing the stem cell thereafter to begin to express some genes, and not others, and to thereby acquire a commitment toward a particular cell lineage. Identifying the genetic factors responsible for this initial differentiation step is a non-trivial problem. Yet scientifically, this inquiry is important in understanding the initial development of living organisms.

It is possible to do comparative RNA analysis between stem cells and cells which have made the first step in differentiation, and identify what species of RNA are produced in the progeny cell that are not produced in the stem cell. A comparative RNA expression study makes it is possible to know what genes are turned on when a cell commits to a specific lineage as compared to the undifferentiated stem cell from which it arose. However, having a catalog of the genes which turn on does not help to distinguish the gene or genes which initiated the process from the larger number of gene which are turned on as a result of the process. In fact, it may be quite difficult or impractical using comparative RNA analysis to identify a gene which initiates the differentiation process since intracellular factors, such as transcription factors, need not be produced in great abundance to have the effect of causing a change in cell differentiation. Proven methods have not been available heretofore to identify the factors which are responsible for primary cell differentiation.

### BRIEF SUMMARY OF THE INVENTION

The present invention is summarized in that a method is described for identifying the cellular factors responsible for cell differentiation from a beginning cell type to a target cell type. The method begins with the random cloning of expressed genes by use of a cDNA library, the cDNA being from the target cell type. The cDNA genes are transferred into expression vectors effective in cells of the beginning cell type. The expression vectors are transferred into the beginning cells and then the cells are cultured in a way so as to permit differentiation into the target cell type. Those cells which have differentiated to the desired target cell type are identified, preferably through the use of a selectable marker. Then the DNA is recovered from the differentiated cells and that DNA is analyzed to determine what inserted cDNA caused the differentiation to the target cell type. In this way, the cellular factors responsible for a specific single cell differentiation can be identified.

It is an object of the present invention to provide the a method to identify the factors responsible for primary cell differentiation.

It is a feature of the present invention that it permits the identification of the genetic factors responsible for the initial stages of cellular differentiation beginning from human embryonic stem cells.

Other objects, advantages, and features of the present invention will become apparent from the following specification.

### BRIEF DESCRIPTION OF THE SEVERAL VIEWS OF THE DRAWINGS

FIGS. 1 and 2 are illustrations of vectors adapted for use in the method of the present invention.

### DETAILED DESCRIPTION OF THE INVENTION

The present invention is intended to identify the genes or genetic factors which are responsible for the primary differentiation of undifferentiated cells to differentiated or partially differentiated cells. The method described here may also be used to identify the genes or genetic factors which cause initially differentiated cells to differentiate further into various cell lineages in the body. The method, which is referred to here as expression cloning, makes use of a gene expression library which is placed in expression vectors and inserted into the undifferentiated cells of interest. Then the undifferentiated cells are permitted to differentiate. Those cells which have differentiated into the cell type of specific interest are then identified. Once the targeted cells have been identified, conventional DNA characterization techniques can be used to identify which cDNA species causes the differentiated cells to differentiate into the cell type of interest.

The present invention was developed to permit the identification of the genetic factors responsible for the first stages of cell differentiation, i.e. to identify those factors which cause the most undifferentiated cells, pluripotent stem cells, to begin the process of differentiation into the various tissues of the body. In particular, using human stem cells, it is possible to use this process to determine what genetic factors cause the stem cells to begin the differentiation process into the various cell lineages which ultimately make up all the cells of the human body. While the techniques and details of the method were developed to permit the application of the process to human embryonic stem cells, it is contemplated that the process can be used at many levels of differentiation with many cells types in culture, from a beginning undifferentiated cell to a terminally differentiated target cell type. The term human undifferentiated stem cell is used here to refer to cells which have the developmental potential of human embryonic stem cells, specifically including cells are derived from other sources such as human embryonic germline cells and stem cells derived from mature or adult bodies.

The process of the present invention thus begins with selecting a beginning cell type, such as an undifferentiated cell type, and a target cell type, such as a cell which has undergone one differentiation step from a stem cell to the precursor of some other type of cell. For purposes of having an example, assume that the target cell type is a cell which has become committed to neural cell lineage, but which is otherwise undifferentiated, a cell type which will be referred to here as a neural precursor cell.

The next step in the process is to select a library of expressed genes from the cells. There are two broad ways to accomplish this objective, to make a library or to use a reference library. Typically, when collections of nucleic acids indicative of gene expression is desired, one uses or makes a cDNA library for the tissue, cell or organism in question. A cDNA library is typically made from the mRNA species which are present in cells in the lineage of the target cell type and is most suitably made from the target cell type itself. The other alternative is to use a reference library collection. For example, a collaborative scientific effort is underway, under the guidance of the U.S. National Institutes of Health, to establish a gene collection to be known as the Mammalian Gene Collection (MGC). The MGC would be a defined gene expression library, intended to overcome some of the limitations in the use of mRNA libraries made for an individual experiment or investigation. The MGC will include clones, identifiers and sequences for the full-length transcripts from mouse and human cDNA libraries. The use of a clone set from a reference library ensures that the clones will be full length, and avoids two common problems with laboratory created mRNA libraries. The problems are that an mRNA library will tend to over-represent the abundant genes expressed in the cell from which the library is made and that the clones in the library will often not be full-length. In general, because of the more fair representation of cDNA species, and the full-length of the members of clones, it is expected that the use of a reference library of expressed genes will generally be more efficient and preferred. The use of the reference library also permits the identification of subsets of clones or genes to preferentially examine desired categories of genes, such as transcription regulators, in preference to other genes or to limit the number of clones which must be created. So, again in the neural precursor cell example, the cDNA library is made by either method to represent the mRNA species present in terminally differentiated nerve cells, neural precursor cells, or any cells located in the lineage between the two.

While this method may be used to detect genes responsible for differentiation, it may be used in a reverse sense as well. If the beginning cell is a differentiated cell, and the target cell is a undifferentiated stem cell, the method can be used to identify genes controlling status of a cell as a stem cell.

Then the cDNA library species are cloned into expression vectors capable of expression in primate undifferentiated stem cells. As it turns out, many mammalian gene expression vectors do not work well in stem cells. Thus the selection of the expression vector is a critical parameter, and will be discussed in more detail below.

The expression vector not only includes the cDNA species to be expressed in the transfected stem cells, it also includes a marker gene system that can be used to detect successful transformants. Such a marker system is needed, as will be appreciated from the discussion below, to identify the cells from the stem cell culture which have undergone the desired differentiation step. Marker gene systems, which can include screenable markers which permit cells to be screened for transformants, or selectable markers which permit a selection agent to select for transformants, permit the identification of transformant cells which express the marker gene. For a screenable marker, such as the green fluorescent protein gene which confers fluorescence on expressing cells, the culture of cells is screened for expression of a detectable phenotype, such as cell fluorescence. For a selectable marker, such as a gene for antibiotic resistance, the culture of cells is exposed to an selection agent, such as an antibiotic, which is toxic to all cells except those expressing the selectable marker gene, in this case one for antibiotic resistance. The use of such marker systems is a common practice in gene transformation processes and many other types and example of markers are known in the art.

It is also preferred that the marker system be under the control of a tissue specific promoter specific to the cell type of the target cell. If, for example, a selectable antibiotic resistance gene is used in a process to identify genes responsible for differentiation to neural precursor cells, the antibiotic resistance gene would be under the control of a tissue specific promoter which only expresses the gene it controls in nerve cells. In this way, the marker will be expressed only when the cell into which it is transformed has differentiated into the target cell type.

Thus the process proceeds as follows. The cDNA library is created and cloned into the expression vector system. The expression vectors, including both the cDNA library and the marker system, are transformed into cells of the cell type. The beginning cell type culture is then cultured. It is preferred that this culture not include other conditions which favor cell differentiation. In fact, it is preferred that the cell culture at this step favors cells remaining undifferentiated. In that way, only cells which are caused to differentiate by the presence of the inserted and expressing cDNA will actually differentiate. The differentiated cells can be detected in a number of ways. One way is simply to examine the cells for morphological change consistent with the desired differentiation step. The preferred way is to use the marker system, which was included in the expression vector for just this purpose. The marker system is used to detect which cells are then expressing the marker system, indicating that the tissue specific promoter driving the marker system has commenced to drive expression. This indicates that the cells have differentiated into the target cells. At this point it should be true that the cDNA species which was transformed into this particular cell or cells was responsible for the differentiation of the beginning cell type into the target cells type. It is now necessary to identify what the cDNA was.

This next step is performed most easily by a PCR reaction. The expression vector has previously been characterized so the 5′ and 3′ flanking regions in the vector around the cDNA segment are known. So DNA is recovered from the differentiated cell or cells and a PCR process in performed on the recovered DNA using primers selected from the flanking regions in the expression vector which lie on either side of the cDNA insert. The product of the PCR process will be amplified DNA extending from one primer to the other and thus extending across the cDNA insert. By sequencing the DNA of the PCR reaction product, the DNA sequence of the cDNA insert that caused the cell differentiation can be determined. Assuming that the cell was transformed only by one expression vector, this will indicate that this single cDNA encodes a protein which, when expressed in an undifferentiated cell, causes the differentiation of that cell toward the target cell type. In other words, this process permits the identification of single genetic factors responsible for single steps of cell lineage differentiation.

Note that this method thus required screening a number of clones to find the clones that were associated with differentiation events. Since screening large numbers of clones can be burdensome, note again that the concept of using a well-defined library makes the overall process more efficient. In a laboratory-made library, the number of full-length clones can vary. Since the screening here is for relatively rare events, the more the library is limited to only include the genes likely to be interest, the shorter the search is likely to be for the gene of interest. With a non-random or defined library, it is possible to start with a library where the number of members in the library is a manageable number. For example, the human genome is thought currently to have only about 50,000 open reading frames. If the clones in the library is even more restricted, to cover only species likely to be involved in control of transcription, for example, the number can be further reduced.

As stated earlier, this expression cloning technique requires the use of a cloning vector which works in the undifferentiated cell type. With regard to the study of human embryonic stem cells, finding an expression vector suitable for expressing foreign genes in human embryonic stem cells has proven to be a non-trivial task. Most expression vectors otherwise useful in mammalian cells do not work at any reasonable degree of efficiency in human embryonic stem cells. It has been found here that there are two expression vectors which will permit the expression of foreign genes in human embryonic stem cells. The two vectors are an Epstein-Barr virus based expression vector and the second type is a Lentivirus expression vector.

The Epstein-Barr virus (EBV) expression vector is based on a commercially available expression vector. The EBV contains a genome of about 172 kb and is maintained in the transformed cells extrachromosomally as a multi-copy, circular episome. The episome replicates with the cells and is faithfully partitioned to daughter cells. It has been found that an EBV vector is capable of transferring into human embryonic stem cells an episome containing an inserted DNA construct which is then faithfully expressed in the transformed stem cells. Further information about the EBV based expression vectors is contained in attachment 1 included with this submission.

Lentivirus vectors are based on the family of retroviruses including human immunodeficiency virus (HIV). Lentivirus vectors have proven efficient at transforming human embryonic stem cells. The lentiviral genome contains the structural genes common to all retroviruses (gag, pol, and env) and in addition contain two regulatory (tat and rev) and four accessory genes (vpr, vif vpu, and nef). The four accessory genes function in replication and pathogenesis in vivo and can be eliminated from lentiviral vectors, although some of these may offer benefit for some cell types for the expression vector function. In the lentiviral vectors contemplated to be used in this process described in this invention, plasmid vectors are used to express gag, pol, tat and rev in a packaging cell line, but intact copies of the genes are eliminated from the actual transfer expression vector transferred into the stem cell lines. The tropism of retroviruses is largely determined by the env protein which binds to specific cell surface receptors. Therefore cells which lack the appropriate cell surface receptor may be difficult to transform with the retrovirus. To confer the broadest possible tropism on the expression vector, the lentiviral vectors will be pseudotyped with the vesicular stomatitis virus (VSV) G glycoprotein. VSV-G interacts directly with the phospholipid component of a cell membrane to mediate viral entry into the cell by fusion with the cell membrane. VSV-G can replace the env protein in retroviruses to produce hybrid pseudotype virus particles with extremely broad tropism. The expression vectors can be derived from vectors which contain cis-acting sequences of HIV required for packaging, reverse transcription, and integration. These sequences include the HIV 5′ LTR, the leader sequence and the 5′ splice donor site, about 360 base pairs of the gag gene, with a restriction endonuclease frame shift mutation preventing translation of gag sequences, 700 base pairs of the env gene containing the Rev-responsive element (RRE) for nuclear export, a 3′ splice acceptor site and the HIV 3′ LTR. To reduce the possible interfering effects of viral sequences on gene expression controlled by an internal promoter, all vectors will also contain a 400 base pair deletion of the U3 region of the 3′ HIV LTR. Because this sequence is copied to the 5′ LTR during a reverse transcription in the subsequent genomic integration, the 5′ LTR promoter/enhancer is rendered non-functional after integration into the host genome. Vectors with this modification are sometimes referred to as self-inactivating.

Lentiviral vectors can infect non-dividing cells, an important attribute for this purpose. The ability of lentiviral vectors to infect non-dividing cells is mediated through the gene products of the gag, pol, and vpr genes. Recently it has been identified that another element involved in nuclear import is a cis-determinant present within the pol coding region, known as the central purine tract (cPPT). For this reason, the cPPT region will be incorporated into the lentiviral vectors for use in this invention. Integration position effects due to the random integration of the retrovirus into the genome contribute to transcriptional silencing of vectors shortly after integration and also contribute to expression variegation and extinction of expression.

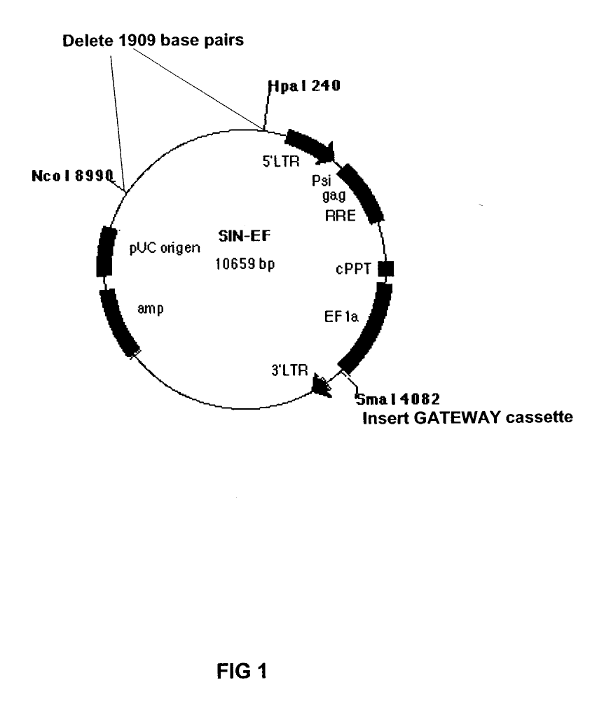

To construct an actual lentivirus vector, we began with a gift lentivirus vector, pSIN-EF-EGFP from Robert Hawley, American Red Cross, Rockville, Md. To modify the vector for use with human embryonic stem cells, the vector was modified to decrease the size of the construct and to make recombinations more efficient. To simplify subsequent cloning steps, the GFP cassette was removed from the vector by digestion with BamHI and religating the vector, the relegated vector being designated pSIN-EF-del. To decrease the size of the vector, 1909 base pairs were deleted from the NcoI site (8990) to the HpaI site (202) to make a vector named pSIN-EF-del2). This decreased the vector size from 10659 to 8750 base pairs. Then the GATEWAY (Invitrogen, Life Sciences, Carlsbad, Calif.) vector conversion cassette B was added to the SmaI site at base pair 4082 in the original vector to make a vector designated pSIN-EF-del2-GATEWAY. This vector can now be used to directly transfer individual clones, groups of clones, or entire libraries to the lentivirus vector for use in over-expression studies. The vector is illustrated in FIG. 1 and the sequence of the vector is contained in SEQ:ID:NO:1.

The development of an EBV-based vector has been carried forward. An EBV vector was acquired from the laboratory of William Sugden, the University of Wisconsin. To modify the vector as received (p2300) for use with human embryonic stem cells, the promoter was changed from the CMV promoter to the EF1 alpha promoter. In addition, a polylinker was added to make the vector easier to manipulate with conventional ligation-mediated cloning procedures. In addition, the GATEWAY cassette was added to the vector to make the vector compatible with the recombination system.

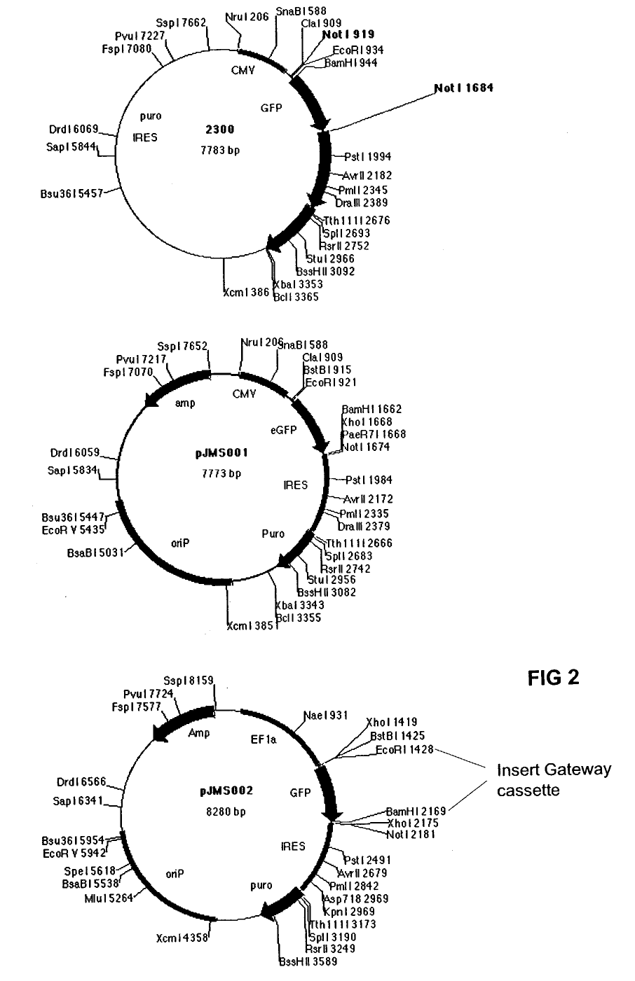

To make these changes, we began by amplifying the gene for green fluorescent protein (GFP) using primers JS 1 and JS2 below, which contain multiple restriction sites. The PCR product was digested with NotI and ClaI and cloned into NotI/ClaI sites of p2300. The resulting plasmid was designated pJMS001. Next the EF1 alpha promoter was amplified with the primers JS5 and JS7 below, the product was digested with SmaI and EcoRI, and the resulting fragment was ligated into the NruI/EcoRI site of pJMS001. The resulting plasmid was named pJMS002. Then pJMS002 was cut with EcoRi and BamHI, and the ends were blunted using T4-DNA polymerase. GATEWAY cassette B was ligated into the plasmid, resulting in a plasmid named pJMS002-GATEWAY. This vector, adapted for use in the method described here, is illustrated in FIG. 2 and its sequence is set forth in SEQ:ID:NO:2.

Primer list
JS1:
GCATCGATTTCGAAGAATTCCACCGGTCGCCACCATGGTG
JS2:
AAAAGGAAAAGCGGCCGCCTCGAGGGATCCTTTACTTGTACAGCTCGTCC
JS5:
CGGCCCGGGGTGAGGCTCCGGTGCCCGTC
JS7:
GGCGAATTCGAACTCGAGACCACGTGTTCACGACACC

I/we claim:

1. A method for identifying a genetic factor responsible for differentiation of a beginning cell to a target cell, the method comprising the steps of
obtaining a cDNA library representing genes expressed in the target cell type;
placing copies of the cDNA library into expression vectors which will express in the beginning cell type;
transforming the expression vectors into cells of the beginning cell type;
culturing cells of the beginning cell type until at least some cells differentiate;
identifying in the cultured cells at least one cell which has differentiated into the target cell type; and
identifying the cDNA in the cell which has differentiated to identify the genetic factor responsible for the cell differentiation

2. A method as claimed in claim 1 wherein the beginning cell type is a human undifferentiated stem cell.

3. A method as claimed in claim 1 wherein the expression vector is selected from the group consisting of an EBV vector and a lentivirus vector.

4. A method as claimed in claim 1 wherein the beginning cell also includes a marker system under the control of a tissue specific promoter, which causes tissue specific expression of the marker system in the target cell type, and further wherein the step of identifying the at least one cell is performed by identifying a cell expressing the marker system.

5. A method as claimed in claim 4 wherein the marker system is a selectable marker.

6. A method as claimed in claim 1 wherein the step of identifying the cDNA is performed by performing a PCR process on DNA recovered from the differentiated cell.

7. A method for identifying a genetic factor responsible for differentiation of a beginning cell to a target cell, the method comprising the steps of
making a cDNA library from mRNA from cells in the lineage of the target cell type;
placing copies of the cDNA library into expression vectors which will express in the beginning cell type;
transforming the expression vectors into cells of the beginning cell type;
culturing cells of the beginning cell type until at least some cells differentiate;
identifying in the cultured cells at least one cell which has differentiated into the target cell type; and
identifying the cDNA in the cell which has differentiated to identify the genetic factor responsible for the cell differentiation.

8. A method as claimed in claim 7 wherein the beginning cell type is a human undifferentiated stem cell.

9. A method as claimed in claim 7 wherein the expression vector is selected from the group consisting of an EBV vector and a lentivirus vector.

10. A method as claimed in claim 7 wherein the beginning cell also includes a marker system under the control of a tissue specific promoter, which causes tissue specific expression of the marker system in the target cell type, and further wherein the step of identifying the at least one cell is performed by identifying a cell expressing the marker system.

11. A method as claimed in claim 10 wherein the marker system is a selectable marker.

12. A method as claimed in claim 7 wherein the step of identifying the cDNA is performed by performing a PCR process on DNA recovered from the differentiated cell.


SEQUENCE LISTING (2 sequences)

SEQ ID NO: 1 - DNA, 10463 bp, Artificial Sequence
  Description of Artificial Sequence vector
gttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccacaaatggcagtattcatccacaattttaaaagaaagggggggattggggggtacagtgcaggggaaagaatagtagacataatagcaacagacatacaaactaaagaattacaaaaacaaattacaaaaattcaaaattttcgggtttattacagggacagcagagatccactttggatcgataagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattcgatatcaagcttatcgatagatctgtcgactaaattctgcagtcgacggtaccgcgggatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatcccgggatccctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattataatagatttagcaggaattgaactaggagtggagcacacaggcaaagctgcagaagtacttggaagaagccaccagagatactcacgattctgcacatacctggctaatcccagatcctaaggattacattaagtttactaacatttatataatgatttatagtttaaagtataaacttatctaatttactattctgacagatattaattaatcctcaaatatcataagagatgattactattatccccatttaacacaagaggaaactgagagggaaagatgttgaagtaattttcccacaattacagcatccgttagttacgactctatgatcttctgacacaaattccatttactcctcaccctatgactcagtcgaatatatcaaagttatggacattatgctaagtaacaaattacccttttatatagtaaatactgagtagattgagagaagaaattgtttgcaaacctgaatagcttcaagaagaagagaagtgaggataagaataacagttgtcatttaacaagttttaacaagtaacttggttagaaagggattcaaatgcataaagcaagggataaatttttctggcaacaagactatacaatataaccttaaatatgacttcaaataattgttggaacttgataaaactaattaaatattattgaagattatcaatattataaatgtaatttacttttaaaaagggaacatagaaatgtgtatcattagagtagaaaacaatccttattatcacaatttgtcaaaacaagtttgttattaacacaagtagaatactgcattcaattaagttgactgcagattttgtgttttgttaaaattagaaagagataacaacaatttgaattattgaaagtaacatgtaaatagttctacatacgttcttttgacatcttgttcaatcattgatcgaagttctttatcttggaagaatttgttccaaagactctgaaataaggaaaacaatctattatatagtctcacacctttgttttacttttagtgatttcaatttaataatgtaaatggttaaaatttattcttctctgagatcatttcacattgcagatagaaaacctgagactggggtaatttttattaaaatctaatttaatctcagaaacacatctttattctaacatcaatttttccagtttgatattatcatataaagtcagccttcctcatctgcaggttccacaacaaaaatccaaccaactgtggatcaaaaatattgggaaaaaattaaaaatagcaatacaacaataaaaaaatacaaatcagaaaaacagcacagtataacaactttatttagcatttacaatctattaggtattataagtaatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgcc

SEQ ID NO: 2 - DNA, 9249 bp, Artificial Sequence
  Description of Artificial Sequence vector
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgggggtgaggctccggtgcccgtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcaagaacacatggtctcgagttcatcaacaagtttgtacaaaaaagctgaacgagaaacgtaaaatgatataaatatcaatatattaaattagattttgcataaaaaacagactacataatactgtaaaacacaacatatccagtcactatggcggccgcattaggcaccccaggctttacactttatgcttccggctcgtataatgtgtggattttgagttaggatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtgtgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacagggactggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgattccctcgaggcggccgcgggcgccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaccgatgcccttgagagccttcaacccagtcagctccttccggtgggcgcggggcatgactatcgtcgccgcacttatgactgtcttctttatcatgcaactcgtaggacaggtgcctggccggggtcccccggaaactcggccgtggtgaccatgcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc